(19) United States
(12) Patent Application Publication
(10) Pub. No.: US 2025/0042938 A1
(43) Pub. Date: Feb. 6, 2025

(54) METHOD FOR THE PURIFICATION OF RECOMBINANT PROTEINS USING OIL BODY PROTEINS

(71) Applicant: CORE BIOGENESIS, ILLKIRCH-GRAFFENSTADEN (FR)

(72) Inventors: Chouaïb MEZIADI, ILLKIRCH-GRAFFENSTADEN (FR); Daniel-Isui AGUILAR-SALVADOR, ILLKIRCH-GRAFFENSTADEN (FR); Michel NEY, ILLKIRCH-GRAFFENSTADEN (FR)

(21) Appl. No.: 18/714,592

(22) PCT Filed: Dec. 1, 2022
(86) PCT No.: PCT/EP2022/083998

§ 371 Date: May 30, 2024

(30) Foreign Application Priority Data
Dec. 1, 2021 (EP) 21306680.6

(51) Int. Cl. C07K 1/32; C07K 14/415

(52) U.S. Cl. CPC C07K 1/32; C07K 14/415

(57) ABSTRACT

The invention relates to a method for the purification of recombinant proteins. Particularly, the present invention relates to a method for the purification of recombinant proteins produced by a first genetically engineered organism by mixing the first genetically engineered organism or part thereof with oil body proteins which are not obtained from the first genetically engineered organism.

### FIELD OF THE INVENTION

The present invention relates to a method for the purification of recombinant proteins. Particularly, the present invention relates to a method for the purification of recombinant proteins produced by a first genetically engineered organism by mixing the first genetically engineered organism or part thereof with oil body proteins which are not obtained from the first genetically engineered organism.

### BACKGROUND PRIOR ART

A recombinant protein is a protein produced by cells whose DNA has been modified by genetic engineering. Recombinant proteins can be molecules of pharmaceutical, therapeutic, cosmetic interest, molecules used in research and development or in the field of agroindustry. Several recombinant protein expression systems using bacteria, yeast, mammalian cells, or other organisms, have been developed. These conventional cell culture-based systems are useful but limited by high cost, low efficiency or low yield. Notably, these systems have an expensive extraction process or, for the most economically attractive ones, do not allow a step of maturation of the recombinant protein which is essential so as to render the recombinant protein functional.

Plants as recombinant protein production hosts have been considered as a viable option. Indeed, they provide a valuable alternative to fermentation-based systems for the production of recombinant proteins, having numerous advantages. Notably, transient expression in plant leaves is one of the most promising technologies for the production of recombinant proteins of interest. In particular, such technology allows for production of mature proteins, i.e. which have been subjected to successful post-translational modifications. However, the extraction of these recombinant proteins relies mainly on the use of tags, making this method undesirable, especially for regulatory reasons. Also, other proteins may interfere with the purification method, making the extraction process less efficient.

U.S. Pat. No. 7,547,821 B2 discloses a method for the expression of insulin in plant seeds comprising the introduction into a plant cell of a chimeric nucleic acid construct which includes a first sequence comprising a seed specific promoter, a second sequence encoding a signal peptide for the secretory pathway and an insulin polypeptide and a third sequence encoding a single chain antibody (scFv) having specificity for an oil body protein. However, such a method does not allow an easy and rapid protein expression as the method requires growing the plant cell into a mature plant capable of setting seed and obtaining substantially pure insulin from said seeds. Such a method requires several months to obtain the recombinant protein, which is a long time when it comes to testing the functionality and efficiency of the produced protein in this system. Also in some cases, a fast and adaptable system allows the molecule of interest's production to be adjusted to the constant changeable need. This is for example the case for vaccines where it is necessary to produce rapidly and flexibly to adapt the epitopes to new variants.

There is therefore a need for a new method or system which allows production of a sufficient amount of recombinant protein in a relatively short time while reducing the cost of goods of the recombinant protein to an affordable price commensurate with the need.

The invention seeks to overcome the aforementioned drawbacks of the prior art as it aims to provide such a method which allows for easy and rapid protein expression and recovery.

### SUMMARY OF THE INVENTION

To this effect the invention discloses a method for the purification of recombinant proteins produced by a first genetically engineered organism, the method comprising the following steps: providing the first genetically engineered organism expressing the recombinant proteins or a part thereof; mixing the first genetically engineered organism or part thereof with oil body proteins which are not obtained from the first engineered organism, wherein the recombinant proteins and/or the oil body proteins are fused with an affinity ligand which allows forming complexes comprising the oil body proteins and the recombinant proteins; recovering the complexes comprising the oil body proteins and the recombinant proteins by flotation of the mix.

Advantageously, the first genetically engineered organism is a genetically engineered plant.

Advantageously, providing the first genetically engineered plant expressing the recombinant protein or a part thereof consists in providing the leaves of the genetically engineered plant.

Advantageously, the genetically engineered plant expressing the recombinant proteins has been engineered by introducing a nucleic construct comprising the coding sequence of the recombinant proteins via agroinfiltration.

Advantageously, the genetically engineered plant or part thereof expressing the recombinant proteins comprises at least one inactivated gene involved in the Transcriptional Gene Silencing (TGS) mechanism and/or at least one inactivated gene involved in the Post Transcriptional Gene Silencing (PTGS) mechanism.

Advantageously, the oil body proteins are selected among a list comprising, or consisting in, caveolin, oleosin, caleosin, steroleosin, perilipin or combinations thereof.

Advantageously, the oil body proteins are oleosin.

Advantageously, the recombinant proteins are selected among a list comprising, or consisting in, insulin, growth factors, therapeutic antibodies, antigens, structural and bioactive peptides or combinations thereof.

Advantageously, the affinity ligand which allows forming complexes comprising the oil body proteins and the recombinant proteins is selected among: a shark affinity ligand, an artificial affinity ligand derived through phage display or bioinformatics or protein predictive models, or combinations thereof.

Advantageously, the affinity ligand which is able to form complexes comprising the oil body proteins and the recombinant proteins is selected among: an antibody or fragment thereof, a single-chain variable fragment (scFv), a sdAb (single-domain antibody), or combinations thereof.

Advantageously, the affinity ligand is a scFv.

Advantageously, the step of mixing comprises mixing the first genetically engineered organism or part thereof with an oilseed plant comprising oil body proteins, preferably with the seeds of an oilseed plant comprising oil body proteins.

Advantageously, the affinity ligand fused with the recombinant proteins and/or with the oil body proteins contains a cleavage site.

Advantageously, the method further comprises a step of purifying the recombinant proteins from the complexes.

Advantageously, purifying the recombinant proteins from the complexes includes modifying the pH to cleave the affinity ligand from the complexes.

Further characteristics, details and advantages of the invention will emerge from the description made with reference to the annexed drawings given by way of example.

### BRIEF DESCRIPTION OF THE DRAWINGS

The invention will be better understood and its various characteristics and advantages will emerge from the following description of a number of exemplary embodiments and its appended figures in which:

FIG. 1 displays the biogenesis of lipid bodies and fused proteins comprising an antibody or fragment thereof or a single-chain variable fragment (scFv) or sdAb with a recombinant protein;

FIG. 2 displays the steps of a particular method of the invention;

FIG. 3 displays an example of a coding sequence for the expression of recombinant proteins fused to scFv by the first genetically engineered organism or part thereof;

FIG. 4 displays an image obtained by phase contrast microscopy and an image obtained with fluorescence microscopy (fluorescein isothiocyanate, FITC-filter) of a mixture comprising oil bodies and a first genetically engineered organism or part thereof comprising a coding sequence for the expression of a recombinant GFP protein fused to an scFv anti-oleosin;

FIG. 5 displays an image obtained by phase contrast microscopy and an image obtained with fluorescence microscopy (using an FITC-filter) of a mix between oil bodies and a GFP proteins;

FIG. 6 displays an image obtained by phase contrast microscopy and an image obtained with fluorescence microscopy (FITC-filter) of oil bodies.

FIG. 7 displays a western blot illustrating the isolation of ScFv-Interleukin 7 fused protein thanks to the method of the invention. The western blot comprises 10 tracks (1: molecular weight markers; 2 and 3: leave extracts comprising ScFv-Interleukin 7 fused protein used as negative control; 4 and 5: mix of leaf extracts comprising ScFv-Interleukin 7 fused protein with wild type lipid bodies; 6: molecular weight markers; 6-10: Interleukin 7 protein at different amounts used as positive control). Corresponding molecular weights (in kDa) are disposed of in regard to the right part of the western blot.

FIG. 8 displays a western blot illustrating the isolation of ScFv-2GF-1 fused protein thanks to the method of the invention. The western blot comprises 6 tracks (1: molecular weight markers; 2: bovine insulin growth factor protein as positive control; 3: mix of leave extracts comprising ScFv-GFP fused protein with wild type lipid bodies, as negative control; 4: leave extracts comprising ScFv-IGF-1 fused protein as a negative control; 5: mix of leave extracts comprising ScFv-IGF-1 fused protein with wild type lipid bodies). Corresponding molecular weights (in kDa) are disposed of in regard to the right part of the western blot.

### DETAILED DESCRIPTION OF THE INVENTION

In this specification, the invention will be described by way of examples, demonstrating reduction to practice of the method of the invention. However, the invention is not restricted to these examples.

The present invention relates to a method for the purification of recombinant proteins produced by a first genetically engineered organism, the method comprising the following steps: providing the first genetically engineered organism expressing the recombinant proteins or a part thereof; mixing the first genetically engineered organism or part thereof with oil body proteins which are not obtained from the first engineered organism, wherein the recombinant proteins and/or the oil body proteins are fused with an affinity ligand which allows forming complexes comprising the oil body proteins and the recombinant proteins; and recovering the complexes comprising the oil body proteins and the recombinant proteins by flotation of the mix.

By “first genetically engineered organism or part thereof”, it is meant any single living plant, animal, algae, yeast or bacteria which has been genetically engineered. The term “genetically engineered organism” is used here to describe an organism that is chromosomally transgenic, or harbours a plasmid or an autonomous replicating sequence, a recombinant virus or viroid or organisms that express genes transiently without the need for integration into the host genome. The first genetically engineered organism or part thereof may therefore be an organism genetically engineered by introducing nucleic acids for transient expression, or may be an organism obtained from a genetically engineered organism, or the like. In particular, the first genetically engineered organism may comprise solely one or more parts (e.g. the leaves of a plant) that are genetically engineered while its others parts remain wild type. This is typically the case when the first genetically engineered organism is a plant expressing the recombinant proteins and has been engineered by introducing a nucleic acid construct comprising the coding sequence of the recombinant proteins via agroinfiltration, as disclosed in more details below.

In the case of an animal organism, the cell culture can be a mammalian cell culture (human or non-human) such as mesenchymal stem cells, embryonic stem cells, induced pluripotent stem cells, hematopoietic stem cells, T-lymphocytes (cart-T) or others. In the case of human embryonic stem cells, these are obtained only without destroying the embryo from which they are derived and are not capable of inducing the developmental process of a human being.

By “expressing the recombinant proteins”, it is meant that the first genetically engineered organism or part thereof harbours a coding sequence for the expression of a recombinant protein.

By “oil body proteins” it is meant proteins or peptides occurring naturally or partly engineered or fully engineered and which have the capability of targeting their localization to oil bodies, i.e. bodies comprising a lipid core and a phospholipid membrane. Accordingly, any animal, human, bacterial, yeast, fungal or plant oil body protein may be used to perform the method of the invention. Because the said oil body proteins are not obtained by the first genetically engineered organism, they are obtained from a second organism, said second organism can be a wild type or genetically engineered. Further, according to such a definition of “oil body proteins”, the method of the invention also corresponds to the use of only a part of known oil body proteins, native or engineered, as long as such part has the capability of targeting its localization to oil bodies.

By “fused with”, it is meant that (1) the first genetically engineered organism or part thereof comprises cells having a nucleic acid construct which includes a coding sequence for a recombinant protein coupled to a coding sequence for an affinity ligand and/or that (2) the oil body proteins are coupled to an affinity ligand. A nucleic acid construct which allows obtaining fusion proteins may comprise coding sequences of proteins which are “linked in a reading frame”, i.e. coding sequences which are set on consecutive triplets of DNA, which are non-overlapping.

By “affinity ligand” it is meant a molecule which is able to bind to a specific target, the specific target being according to the invention (1) a recombinant protein and/or (2) an oil body protein, as long as the affinity ligand which is either fused to a recombinant protein or an oil body protein allows forming complexes comprising the oil body protein and the recombinant protein.

By “wherein the recombinant proteins and/or the oil body proteins are fused with an affinity ligand”, it is not meant only that multiple recombinant proteins are fused to one unique affinity ligand or that multiple oil body proteins are fused to one unique affinity ligand. One recombinant protein may be fused to one affinity ligand and one oil body protein may be fused to one affinity ligand.

By “wherein the recombinant proteins and/or the oil body proteins are fused with an antibody or fragment thereof or a single-chain variable fragment (scFv) or a single-domain antibody (sdAb)”, it is meant that multiple recombinant proteins may be fused to one unique antibody or fragment thereof or scFv or sdAb or that multiple oil body proteins may be fused to one unique antibody or fragment thereof or scFv or sdAb. One recombinant protein may be fused to one antibody or fragment thereof or one scFv or one sdAb and one oil body protein may be fused to one antibody or fragment thereof or one scFv or one sdAb.

By “allowing forming complexes” it is meant that (1) the affinity ligands fused with the recombinant proteins are able to target and bind to the oil body proteins mixed, via the affinity ligands, and/or (2) the affinity ligands fused with the oil body proteins are able to target and bind to the recombinant proteins, via affinity ligands. Therefore it is meant that a complex as in the scope of the present invention comprises at least an oil body protein, a recombinant protein and an affinity ligand fused to one of the said oil body protein or said recombinant protein and bound to the other one.

By “a single-chain variable fragment (scFv)”, it is meant a fusion protein of the variable regions of heavy and light chains of immunoglobulins, connected with a linker peptide of 10 to 25 amino acids.

By “a single-domain antibody (sdAb)”, it is meant an antibody fragment consisting of a single monomeric variable antibody domain, which are also known as camelid, nanobodies or camelid nanobodies.

By “flotation” it is meant that the mix is introduced into a liquid solution, such as a buffer at pH 7, a phosphate-buffered saline solution or the like, and is centrifuged. In the present invention, after this step, the oil bodies, which float to the surface, may be recovered, as they now contain the complexes comprising the oil body proteins and the recombinant proteins.

The method of the present invention comprises mixing the first genetically engineered organism or part thereof with oil body proteins which are not obtained from the first engineered organism but from a second organism. Using oil body proteins which are not obtained from the first engineered organism avoids the need of growing such organism until obtaining the oil body proteins (e.g. until obtaining the seeds) and therefore allows considerably reducing the time required for the production of recombinant proteins which are fully functionalized, notably with the correct protein folding. Notably, a step of growing a genetically engineered organism is a long process which considerably slows the process for obtaining recombinant proteins.

The recombinant protein may be related to proteins used in cosmetic industry, pharmaceutical industry, food industry or agriculture. Advantageously, the recombinant protein is selected among growth factors, interleukins, cytokines enzymes, structural proteins, receptors and signaling proteins, antibodies and antigens.

In the case that the first genetically engineered organism is a plant, the plant is preferably selected among Nicotiana spp including Nicotiana benthamiana. A genetically engineered plant being a double mutant rdr2rdr6 of the Nicotiana benthamiana species is particularly preferred as the first genetically engineered organism. It is particularly advantageous to perform the method with a first genetically engineered organism which is a plant as the proteins are soluble. In addition, providing a plant or part thereof for mixing with oil body proteins may not require any intermediary step of preparation, which allows recovering the recombinant proteins in an easy and fast manner.

Furthermore, in the case that the first genetically engineered organism which is a plant expressing the recombinant proteins has been engineered by introducing a nucleic acid construct comprising the coding sequence of the recombinant proteins via agroinfiltration or other means to induce transient expression of the construct, the expression of the recombinant proteins may be carried out within few days or less and therefore the method of purification of the invention may be performed as soon as the quantity of recombinant proteins expressed and functionalized is considered sufficient. In one embodiment which may be combined with any other embodiment disclosed herein, leaves of a genetically engineered plant are used as they may have been genetically engineered for transient expression. Protein expression in leaves is particularly suited for certain recombinant proteins and allows maintaining a plant growing while carrying out the method on only the leaves of a plant.

By “agroinfiltration”, it is meant in the invention a method to induce transient expression of genes in a plant, or isolated leaves from a plant, or even in cultures of plant cells, in order to produce a desired protein. In the agroinfiltration method, a suspension of Agrobacterium tumefaciens is introduced into a plant leaf by direct injection or by vacuum infiltration, or brought into association with plant cells immobilised on a porous support (plant cell packs), whereafter the bacteria transfer the desired gene into the plant cells via transfer of T-DNA. The main benefit of agroinfiltration when compared to the more traditional plant transformation is speed and convenience, although yields of the recombinant protein are generally also higher and more consistent.

In a particular embodiment, which may be combined to any other embodiment wherein the first genetically engineered organism is a plant, the genetically engineered plant expressing the recombinant proteins comprises at least one inactivated gene involved in the Transcriptional Gene Silencing (TGS) and/or at least one inactivated gene involved in the Post Transcriptional Gene Silencing (PTGS) mechanism.

Alternatively or in a complementary way, the nucleic acid construct may comprise at least one gene suppressor of the Transcriptional Gene Silencing (TGS) and/or at least one gene suppressor of the Post Transcriptional Gene Silencing (PTGS) mechanism. Notably, the at least one gene suppressor of the Transcriptional Gene Silencing (TGS) and/or the at least one gene suppressor of the Post Transcriptional Gene Silencing (PTGS) has a transient expression.

By “gene involved in the Transcriptional Gene Silencing (TGS) and/or Post Transcriptional Gene Silencing (PTGS) mechanism”, it is particularly understood as any gene involved in the TGS and/or PTGS mechanism which would prevent the production of the recombinant protein. By “at least one inactivated gene” as used herein it is meant a gene which cannot encode RNA or a protein or encodes no functional protein, or which encodes a protein with reduced activity by at least 90%. Said “inactivated gene” can encode a protein with reduced functionality, or be a fully inactivated gene, encoding no functional protein or not encoding any protein. An inactivated gene involved in the TGS or PTGS mechanism in the present invention is usually a wild-type gene, i.e. a wild-type gene which, due to the genetically engineered inactivation, cannot encode an RNA or protein, or encodes no functional protein, or encodes a protein with reduced activity, by at least 90%. A inactivated gene may be obtained by genetic alteration, i.e. at least one among the following modifications: an insertion of a stop codon, an insertion of a nucleotide resulting in the creation of a stop codon, a shift of the nucleotide reading frame resulting in a protein which is not functional, the introduction of a recombination site, such as Cre-Lox, or any modification which has a highly suppressive effect of at least 90% on the expression of the target gene. Other modifications altering the coding sequence of the gene so as to inactivate it out may also be used. Such genetic alterations result from the use of a method selected among CRISPR-Cas 9, synthetic RNAi, TALENS, Meganuclease, TILLING and Zinc finger nuclease but other methods may be suitable. As an alternative, other methods such as physical and chemical mutagenesis by Gamma irradiation or EMS treatment may be used. Preferably, the method used for generating the genetic alterations is CRISPR-Cas9.

Advantageously, the “at least one inactivated gene involved in the TGS or PTGS mechanism” is chosen among the Dicer-like (DCL) family, the Suppressor of Gene Silencing (SGS) family, the Argonaute (AGO) family, the RNA dependent RNA polymerase (RdR) family and combinations thereof.

Advantageously, the “at least one inactivated gene involved in the TGS or PTGS mechanism” includes at least one inactivated gene involved in the TGS mechanism and at least one inactivated gene involved in the PTGS mechanism. For instance, the at least one gene involved in the TGS mechanism may be RDR2 or DCL3 or one of AGOs genes. For instance the at least one gene involved in the PTGS mechanism may be selected from RDR6 or DCL2 or DCL4. Advantageously, the “at least one inactivated gene involved in the TGS and/or PTGS mechanism” includes the RdR2 gene and the RdR6 gene.

The oil body protein means any lipophilic protein capable of anchoring in a lipid body used in the method of the present invention. It may be selected among a list comprising caveolin, oleosin, caleosin, steroleosin, caleosin, perilipins or combinations thereof. Preferably, the oil body protein is an oleosin, as it is, among other things, more conserved through the species.

In the method of the present invention, the step of mixing may use an oilseed plant as it contains more oil body proteins. Preferably, the seeds of an oilseed plant are used for the step of mixing as the seeds typically contain more oil body proteins than other parts of a plant, which would allow forming a greater number of complexes for the recovery of the complexes. Furthermore, for plants which make oil bodies in vegetative structures, including plants engineered for this purpose, non-seed derived oil bodies may be used for the method of the present invention.

In a particular embodiment which may be combined to the other embodiments, the first genetically engineered organism comprises cells comprising a coding sequence for a peptide signal, such as the PR1b signal peptide, in reading frame with the coding sequence for a recombinant protein and an affinity ligand, such as for instance an antibody or part thereof or a scFv or a sdAb. The PR1b signal peptide allows specifically directing the fused protein which would be expressed from the coding sequence to follow the apoplast pathway, i.e. through the cells rather than the symplast pathway, i.e. through the cytoplasm. In particular, the complexes are less likely to be affected by the proteases in the apoplast pathway, which therefore does not reduce the amount or recombinant protein which may be recovered when the method of the invention is carried out.

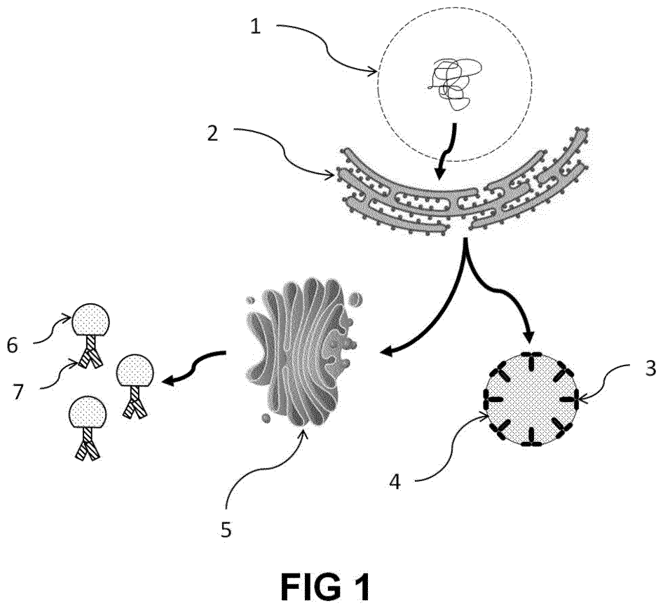

FIG. 1 displays the biogenesis of lipid bodies and fused proteins comprising an affinity ligand fused with a recombinant protein. In FIG. 1, the nucleus 1 of a cell of the first genetically engineered organism comprises the coding sequence for a recombinant protein in reading frame with an affinity ligand. When expressing such coding sequence, the complete biogenesis for the fused proteins follows the protein biosynthesis process from the nucleus 1, through the endoplasmic reticulum 2 and the Golgi apparatus 5. A fused protein in FIG. 1 comprises the affinity ligand 7 and the recombinant protein part 6. Lipid droplets 4 are also produced in cells and comprise lipid bodies which are useful for energy metabolism, membrane synthesis or the production of essential lipid-derived molecules. Oil body proteins 3 are also produced by the cells and are structural proteins which cover lipid droplets.

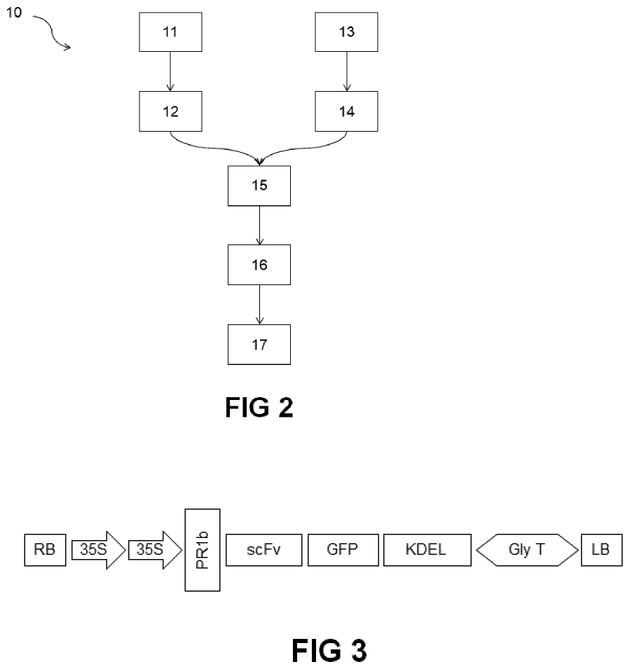

FIG. 2 displays a particular method 10 of the invention, comprising providing 11 a first genetically engineered organism being preferably a plant, the first genetically engineered organism expressing fused proteins comprising recombinant proteins fused with antibodies or fragment thereof or scFv or sdAb; preparing 12 the first engineered organism or part thereof for the mixing step 15, for instance by blending a part of the first engineered organism, especially the leaves in the case of a plant; providing 13 a source of oil body proteins which is not obtained from the first genetically engineered organism, such as for example Camelina sativa seeds; if necessary passing 14 the source of oil body proteins through a colloid mill or other form of homogenization; mixing 15 the first genetically engineered organism or part thereof with the source of oil body proteins which are not obtained from the first engineered organism; centrifuging 16 the mix obtained at the end of step 15; recovering 17 the supernatant comprising the complexes comprising the oil body proteins and the recombinant proteins. The method may be completed by a further purification of the supernatant to isolate the complexes formed with the oil body proteins, the recombinant proteins and the antibodies or fragment thereof or scFv or sdAb.

FIG. 3 displays an example of a coding sequence for the expression of recombinant proteins fused to a scFv by the first genetically engineered organism or part thereof, the coding sequence comprising from the left of the figure to the right: a right border (RB), a tandem 35S promoter, a PR1b signal peptide, an Open Reading Frame encoding a single-chain Fragment variable (scFv) antibody anti oleosin, a GFP marker gene, a KDEL signal retention peptide, a Terminator Glycine (Gly T) and a left border (LB). Accordingly, and when the first genetically engineered organism comprises such coding sequence, the method of the present invention, and particularly the method as described in the paragraphs describing FIG. 2, the scFv-GFP fusion protein is produced, for instance in Nicotiana benthamiana leaves. These leaves, which may have been blended previously, are then mixed with Camelina sativa seeds which may have been milled through a colloid mill or homogenizer previously. The oil body proteins, here oleosins, are recognized by the scFv and therefore bind together, forming complexes of an oil body protein with a scFv which is fused to a recombinant protein being the GFP. Therefore, the oil body proteins on the surface of lipid bodies may be observed by fluorescence.

The PR1b signal peptide (pathogenesis related protein 1) is a signal peptide which allows the recombinant proteins fused to an antibody part thereof or scFv to follow the secretory pathway to the apoplast. In particular, directing the fused proteins allows avoiding the degradation of the fused proteins by proteases which are more present in the cytoplasm or symplast.

The KDEL signal retention peptide is a signal peptide which corresponds to the sequence for the amino acids K-D-E-L. The KDEL signal retention peptide ensures the targeting of the recombinant protein fused to an affinity ligand to the endoplasmic reticulum and retaining it. Further, the KDEL signal retention peptide allows retaining the recombinant protein in the endoplasmic reticulum, which prevents the degradation of proteins and increases their accumulation.

In an example of the present invention, scFv-GFP fusion proteins in Nicotiana benthamiana leaves may be produced following agroinfiltration, the scFv targeting oleosins. After the transformed cells of the leaves have expressed the coding sequence for the fused proteins, these leaves may be blended and mixed with Camelina sativa seeds. The scFv allows binding with the oleosins and the formation of complexes. The complexes are therefore bound to the oil bodies, allowing the recovery of the recombinant proteins by centrifugation and recovery of the supernatant.

Previous oleosin fusion systems did not allow for the glycosylation of the recombinant proteins of interest because the trafficking of the lipid bodies occurs between the endoplasmic reticulum leaflet and the cytoplasmic membrane. In one aspect of the present invention, this lack of glycosylation is remedied as the antibody or part thereof or scFv is bound to the recombinant protein and follows the secretory pathway through the Golgi apparatus, therefore allowing glycosylation of the recombinant protein of interest.

### Example 1: Agroinfiltration

Agroinfiltration for obtaining transformed leaves with a nucleic acid construct comprising the fused protein of interest was carried out as follows.

First, a box was filled with soil (NF U 44-451, Jardiland) and watered abundantly. Seeds of the Nicotiana benthamiana plant were sown directly into the soil. The box was then partially closed and placed in a culture chamber with a photoperiod of 16/8 of day/night with humidity at 70% and at a temperature of 24° C.

After 10 to 14 days, seedlings were transferred into individual pots. Soil was first sterilised at 80°° C. for 3 hours before its introduction into the pots. Then, the soil was watered abundantly. The box was also watered abundantly before seedlings were individually transferred into one pot.

Seedlings were then grown during 42 days in the culture chamber.

Two days before carrying out agroinfiltration step, cultures of A. tumefaciens bacteria (strain C058 or EHA105) with the nucleic acid construct comprising the fused protein of interest were seed into 5 mL of Lysogeny broth (LB) culture medium with the appropriate antibiotics to the construct (e.g. Gentamicin and Kanamycin in case of p35SD or pBin promoters in the acid nucleic construct). The bacterial cultures were agitated overnight at 250 rpm at 28° C.

The next day, the bacterial cultures were disposed into an Erlenmeyer flask of 2 L with 500 mL of LB culture medium comprising the appropriate antibiotics to the construct. Bacterial cultures were agitated until the next day at 230 rpm at 28° C. The next day, bacterial cultures were centrifuged at 5000 g during 15 minutes.

Meanwhile, an agroinfiltration solution was prepared with 10 mM of MED-hydrate (Sigma-Aldrich, M8250), 10 mM of MgCl2-Hexahydrate (Sigma-Aldrich, M9272), and 500 μM of acetosyringone (Acros Organics, 115540050), at pH 5.8.

Then, the pellet of centrifuged bacteria was re-suspended into the agroinfiltration solution for obtaining a finalized agroinfiltration solution.

Each individual grown plant was placed within the housing of a vacuum pump, with most of the leaves soaking into the finalized agroinfiltration solution, pot upside. Vacuum of the housing was then carried out to reach from 0.95 to 1 bar, and hold for 1 minute.

The plants were afterwards placed again in the culture chamber to let the transformed leaves grow up.

Transformed leaves were then harvested and placed at 4° C. before being stored at −80° C. for later extraction.

### Example 2: ScFv-GFP Fused Protein

As proof of concept of purification of fused proteins of interest according to the method of the invention, the inventors used two transformed Nicotiana benthamiana leaves.

First Nicotiana benthamiana plant with transformed leaves able to produce recombinants ScFv-GFP fused proteins was obtained according to the agroinfiltration protocol of Example 1. The related nucleic acid construct comprised the different parts illustrated in FIG. 3, wherein the scFv targets oleosin and wherein it further comprises transient co-expression of a suppressor of PTGS from the potato virus Y (HcPro; Brigneti et al., 1998).

Second Nicotiana benthamiana plant with transformed leaves able to produce recombinants GFP protein was obtained according to the agroinfiltration protocol of Example 1. The related construct comprised the different parts illustrated in FIG. 3 except the scFv sequence and wherein it further comprises the HcPro sequence.

Then for the first and second transformed leaves, 500 mg of transformed leaves were frozen and grinded by homogenization in 1 ml of PBS with 4% polyvinylpolypyrrolidone (PVPP). Subsequently extracts of leaves were obtained by filtration through miracloth (Millipore, ref: 475855).

Besides, lipid bodies were obtained from wild type Camelina sativa seeds. 1 g of seeds was soaked in a buffer of Tris 20 mM pH8, NaCl 0.5 M for 1 h, and then grinded in the glass. The final volume was 50ml. This was separated in two falcon tubes and centrifuged at 4 k rpm for 90 min at 4° C. Lipid bodies' fraction was removed and transferred to ten Eppendorf tubes of 2 ml, centrifuged at 13.3 k rpm, 4° C. for 25 min. Said process was repeated until the final resuspension volume was 2 ml. The samples were stored at 4° C.

For each first and second leave's extract, 50 μL of leave's extract were incubated with 100 μL of lipid bodies and 1 ml of Tris-HCl 20 mM at pH 7,5 for 3 hours at 28° C. under agitation. Then the solutions were split in two fractions and centrifuged using an Eppendorf centrifuge at 4K rpm for 90 min at 4° C. The lipid bodies' fractions were retrieved into 2 ml tubes and the fraction was washed 3 times in PBS until a final resuspension in 1 ml.

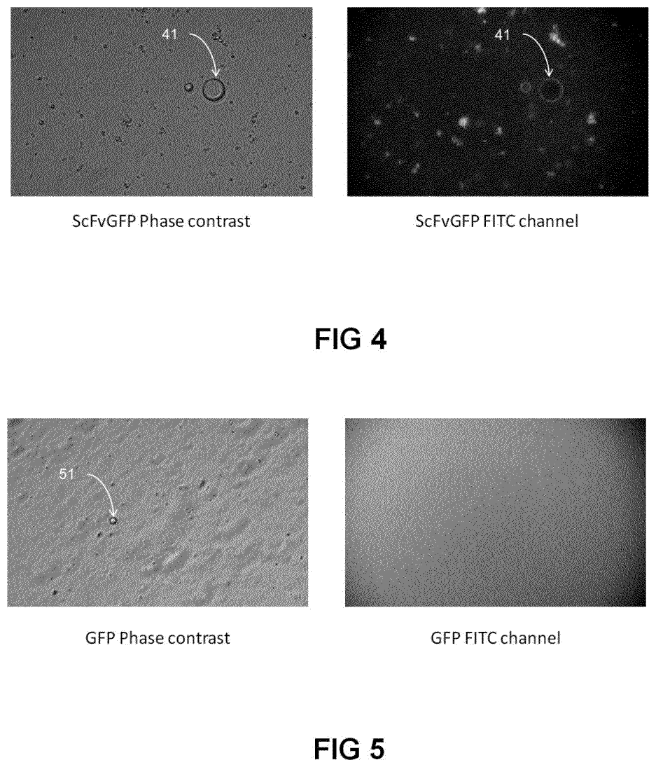

FIG. 4 displays an image obtained by phase contrast microscopy and an image obtained with fluorescence microscopy (fluorescein isothiocyanate, FITC-filter) of a mix between oil bodies and a first genetically engineered organism or part thereof comprising a coding sequence for the expression of a recombinant GFP protein fused to a scFv anti-oleosin. FIG. 4 displays what can be observed with the example of a coding sequence as described in the paragraph describing FIG. 3 and with the method as described in the paragraph describing FIG. 2. On the left part of FIG. 4, which corresponds to an image obtained by phase contrast microscopy, an oil body 41 can be observed, oil body proteins being present at the surface. On the right part of FIG. 4, which corresponds to an image obtained by the use of fluorescein isothiocyanate (FITC) filter, the same oil body protein 41 is visible due to the fluorescence of the GFP which is specific to the corona of large lipid bodies and bright spots in lipid bodies. Such microscopy images demonstrate that complexes between the fusion protein comprising the scFv anti-oleosin and GFP and oleosins have been formed.

FIG. 5 displays an image obtained by phase contrast microscopy and an image obtained with fluorescence microscopy (fluorescein isothiocyanate, FITC-filter) of a mix between oil bodies and a GFP protein. FIG. 5 displays what can be observed when mixing oil bodies with GFP instead of the fusion protein comprising the scFv anti-oleosin and GFP. Lipid body 51 and other smaller lipid bodies can be observed by phase contrast microscopy. However, fluorescence microscopy only reveals any dispersed fluorescence, indicating that no complexes between the oil body proteins and GFP were formed.

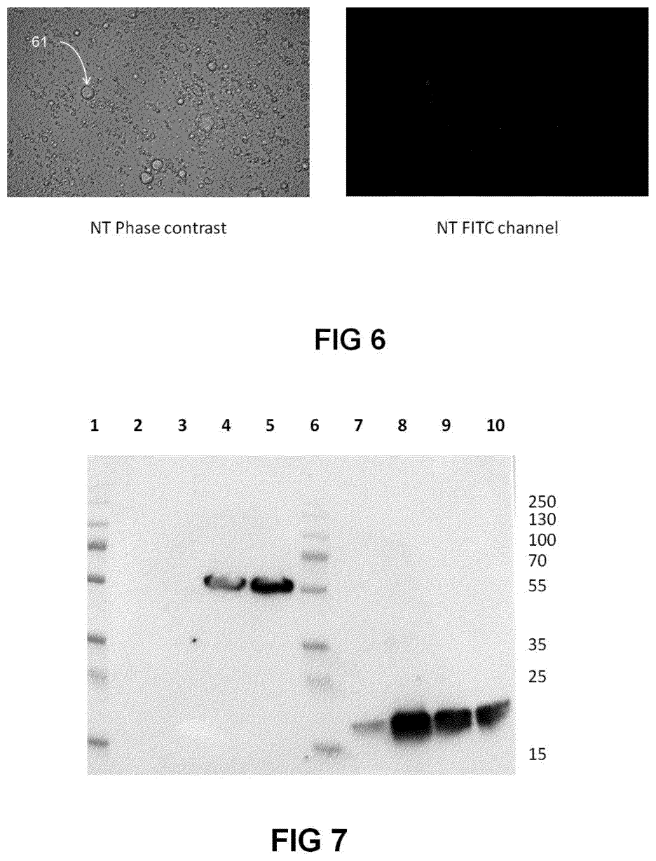

FIG. 6 displays an image obtained by phase contrast microscopy and an image obtained with fluorescence microscopy (FITC-filter) of oil bodies. FIG. 6 displays oil bodies through phase contrast microscopy and fluorescence microscopy without any GFP or scFv, as a control test. Lipid body 61 can be observed by phase contrast microscopy. However, fluorescence microscopy does not reveal any fluorescence, indicating that the GFP is not present and that the fluorescence observed in FIGS. 5 and 6, regarding fluorescence microscopy, corresponds to the GFP.

### Example 3: ScFv-Interleukin 7 Fused Protein

In this example, the inventors succeeded to purify a ScFv-Interleukin 7 fused protein.

### Material and Method

Two Nicotiana benthamiana plants with transformed leaves able to produce recombinants ScFv-Interleukin 7 fused proteins were obtained according to the agroinfiltration protocol of Example 1. The constructs include the same parts as the one illustrated in FIG. 3 wherein the scFv targets oleosins notwithstanding that for the first and second transformed plants the GFP sequence was replaced by an interleukin 7 sequence and that they also included the HcPro sequence. Moreover for the second transformed plant, the KDEL signal retention was absent.

Plant leaves extracts of both transformed plants were recovered as described in Example 2.

Besides, lipid bodies were obtained according to the same protocol as mentioned in Example 2.

From the leaves extracted from each first and second transformed plant, two preparations were prepared. In a first preparation, 40 ml of leaves extracts were incubated with 8 ml of lipid bodies for 2 h at 4° C.

The second preparation corresponded to raw leaf extract.

Then flotation step was carried out on both preparations by centrifugation using an Eppendorf centrifuge at 4K rpm for 90 min at 4° C. The lipid bodies' fraction was retrieved into 2 ml tubes and the fraction was washed 3 times in PBS until a final resuspension in 1 ml.

A first purified solution obtained from the first preparation of the first transformed plant contained 58.95 ng of fused proteins. A first purified solution obtained from the first preparation of the second transformed plant contained 112.474 ng of fused proteins.

A Western blot was carried out with a Bio-Rad Kit (EveryBlot Blocking Buffer 500 mL, ref: 12010020; Trans-Blo Turbo Mini PVDF Transfer Packs, ref: 1704156; 4-20% MP TGX Stain-Free Gel 10W 50 μl×10, ref: 4561094; 10×Tris/Glycine/SDS 5L, ref: 1610772; 25ML Laemmli SDS sample buffer, reducing (6X), ref: 15493939; page Ruler Prest Plus, ref: 26620).

The western blot comprised 10 wells, wherein the first well the first and sixth wells were injected with 3 μL of molecular weight marker from the Kit. In the second and third wells were injected respectively 20 μL of second purified solutions from the second preparation from each first and second transformed plant. In the seventh to the tenth wells, different volumes of Interleukin 7 protein (Preprotech AF200-07) at 100 ng/μL were injected as positive controls: 0.25 μL, 2 μL, 1.5 μl and 1 μL respectively. In the fourth and fifth wells were respectively injected 20 μL of the recovered protein from the first and second transgenic plant, corresponding to 2.95 ng for the first transformed plant and 5.62 ng for the second transformed plant.

The western blot was incubated with an anti-IL7 antibody (Preprotech 500-M07, dilution 1/10000) as a primary antibody at 4° C. overnight. Then the western blot was incubated with an anti-mouse antibody (INVITROGEN A10551) as a secondary anti-body during 1 hour at room temperature.

### Results

FIG. 7 shows the results.

As expected, second and third tracks do not highlight any protein since ScFv-Interleukin 7 proteins are in too low concentration in absence of their mixing with lipid bodies. Besides, positive control tracks highlight proteins at 17.5 kDa corresponding to the molecular weight of the Interleukin 7 protein. The fourth and fifth tracks highlight proteins at 44.5 kDav (17.5 +27) corresponding to the molecular weight of the fused protein, demonstrating the purification of said fused protein for both transformed plants. Moreover, said experiment shown that KDEL peptide signal absence in the second transformed plant is not impairing with the production and recovery of the fused protein.

### Example 4: ScFv-IGF-1 Fused Protein

In this example, the inventors succeeded to purify a ScFv-IGF-1 fused protein.

### Material and Method

Nicotiana benthamiana with transformed leaves able to produce recombinants ScFv-IGF fused proteins was obtained according to the agroinfiltration protocol of Example 1. The construct included the same parts as the one illustrated in FIG. 3 wherein the scFv targets oleosins notwithstanding that it also includes the HcPro sequence and the GFP sequence is replaced by a bovine insulin like-growth factor sequence.

Plant leaves extract was recovered as described in Example 2.

Besides, lipid bodies were obtained according to the same protocol as mentioned in Example 2.

Two preparations were then prepared from the leave's extract as described in Example 3.

Then the flotation step was carried out on both preparations as described in Example 3.

In a first purified solution, obtained from the first preparation, ScFv-IGF-1fused proteins at 8 ng/μL were recovered. A second purified solution from the second preparation was also obtained.

A western blot was carried out with a Bio-Rad Kit (EveryBlot Blocking Buffer 500 mL, ref: 12010020; Trans-Blo Turbo Mini PVDF Transfer Packs, ref: 1704156; 4-20% MP TGX Stain-Free Gel 10W 50 μl×10, ref: 4561094; 10×Tris/Glycine/SDS 5L, ref: 1610772; 25 ML Laemmli SDS sample buffer, reducing (6X), ref: 15493939; page Ruler Prest Plus, ref: 26620).

The western blot comprised 6 wells, wherein the first well was injected with 3 μL of molecular weight marker from the Kit. In the second well, 200 ng of bovine insulin like-growth factor protein (Peprotech 100-11) as a positive control. In the third well, 25 μL of the lipid bodies' fraction of the first transformed leaves (ScFv-GFP fused protein construct) of Example 2, as a negative control. In the fourth well were injected 25 μL of the second purified solution. In the fifth well 25 L of the first purified solution was injected.

The western blot was then incubated with an anti-IGF-1 antibody (Peprotech 500-P11, dilution 1/5000) as a primary antibody at 4° C. overnight. Finally, the western blot was incubated with an anti-rabbit antibody (INVITROGEN A27036) as a secondary antibody during 1 hour at room temperature.

### Results

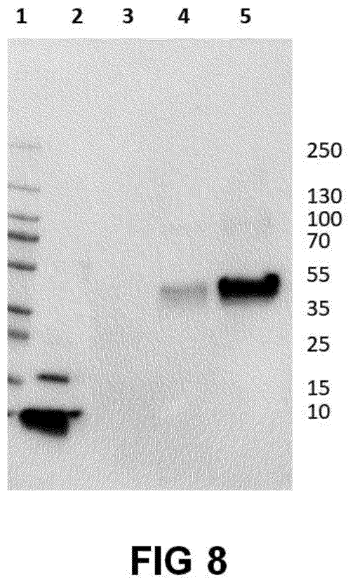

As shown on FIG. 8 and as expected, negative control tracks do not highlight any protein and the positive control track highlight proteins at 7.6 kDa corresponding to the molecular weight of the bovine IGF-1. The fifth track highlights proteins at 36 kDa corresponding to the molecular weight of the fused protein. In the fourth track, few fused proteins were also highlighted but as traces as expected since they were present at very low concentration due to the absence of mixing step with lipid bodies.

1. A method for the purification of recombinant proteins produced by a first genetically engineered organism, the method comprising the following steps;
Providing the first genetically engineered organism expressing the recombinant proteins or a part thereof;
Mixing the first genetically engineered organism or part thereof with oil body proteins which are not obtained from the first engineered organism, wherein the recombinant proteins and/or the oil body proteins are fused with an affinity ligand which allows forming complexes comprising the oil body proteins and the recombinant proteins;
Recovering the complexes comprising the oil body proteins and the recombinant proteins by flotation of the mix.

2. The method of claim 1, wherein the first genetically engineered organism is a genetically engineered plant.

3. The method of claim 2, wherein providing the first genetically engineered plant expressing the recombinant protein or a part thereof consists in providing the leaves of the genetically engineered plant.

4. The method of claim 2, wherein the genetically engineered plant expressing the recombinant proteins has been engineered by introducing a nucleic acid construct comprising the coding sequence of the recombinant proteins via agroinfiltration.

5. The method of claim 2, wherein the genetically engineered plant or part thereof expressing the recombinant proteins comprises at least one inactivated gene involved in the Transcriptional Gene Silencing (TGS) and/or at least one inactivated gene involved in the Post Transcriptional Gene Silencing (PTGS) mechanism.

6. The method of claim 1, wherein the oil body proteins are selected among a list comprising caveolin, oleosin, caleosin, steroleosin, perilipin or combinations thereof.

7. The method of claim 1, wherein the oil body proteins are oleosin.

8. The method of claim 1, wherein the recombinant proteins are selected among a list comprising insulin, growth factors, therapeutic antibodies, antigens, structural and bioactive peptides or combinations thereof.

9. The method of claim 1, wherein the affinity ligand which allows forming complexes comprising the oil body proteins and the recombinant proteins is selected among: a shark affinity ligand, an artificial affinity ligand derived through phage display or bioinformatics or protein predictive models, or combinations thereof.

10. The method of claim 1, wherein the affinity ligand which is able to form complexes comprising the oil body proteins and the recombinant proteins is selected among: an antibody or fragment thereof, a single-chain variable fragment (scFv), a sdAb (single-domain antibody), or combinations thereof.

11. The method of claim 10, wherein the affinity ligand is a scFv.

12. The method of claim 1, wherein the step of mixing (15) comprises mixing the first genetically engineered organism or part thereof with an oilseed plant comprising oil body proteins, preferably the seeds of an oilseed plant comprising oil body proteins.

13. The method of claim 1, wherein the affinity ligand fused with the recombinant proteins and/or with the oil body proteins contains a cleavage site.

14. The method of claim 1, wherein the method further comprises a step of purifying the recombinant proteins from the complexes.

15. The method according to claim 14, wherein purifying the recombinant proteins from the complexes includes modifying the pH to cleave the affinity ligand from the complexes.
